(19) United States
(12) Patent Application Publication
(10) Pub. No.: US 2026/0083318 A1
(43) Pub. Date: Mar. 26, 2026

(54) METHOD AND DEVICE FOR VISUAL FIELD TESTING

(71) Applicant: THE HONG KONG POLYTECHNIC UNIVERSITY, Hong Kong (CN)

(72) Inventors: Xiaolin MEI, Hong Kong (CN); Ming Yan CHEONG, Hong Kong (CN); Lok Hin CHAN, Hong Kong (CN); Lai Lin TSANG, Hong Kong (CN); Lilin CHEN, Hong Kong (CN); Bik Chi SUEN, Hong Kong (CN)

(21) Appl. No.: 19/277,927

(22) Filed: Jul. 23, 2025

(30) Foreign Application Priority Data
Sep. 25, 2024 (HK) 32024097253.1

(51) Int. Cl. A61B 3/024; A61B 3/00

(52) U.S. Cl. CPC A61B 3/024; A61B 3/0058; A61B 3/0091

(57) ABSTRACT

A method and a device for visual field testing are provided. The method is applied to a piece of display equipment which includes a display for presenting a detection interface. Within a preset first duration, a central stimulus point is displayed on the detection interface. Within a preset second duration, a central target and a peripheral target are displayed on the detection interface based on a preset target display strategy. In response to a subject's first trigger operation on the detection interface, a first response result corresponding to the first trigger operation and a target position area of a next round of display of the peripheral target are determined. Further, a testing result of the subject is determined based on the first response results corresponding to a preset number of detections.

### CROSS-REFERENCE TO RELATED APPLICATION

Pursuant to 35 U.S.C. § 119 and the Paris Convention, this application claims the benefit of Hong Kong SAR Short-term Patent No. 32024097253.1 filed on Sep. 25, 2024, the content of which is incorporated herein by reference.

### TECHNICAL FIELD

The present application relates to the field of medical testing technology, more particularly to a method and a device for visual field testing.

### BACKGROUND

The statements provided herein are merely background information related to the present application, and do not necessarily constitute any prior arts. Visual field testing is an important method for evaluating a spatial range that a subject's eyes can see. The result of visual field testing can reflect the degree of difference between the subject's visual field and the normal visual field, which is of great significance for diseases such as glaucoma, retinopathy, head trauma, nervous system diseases and stroke.

In related technologies, the visual field testing is usually performed by professional doctors and ophthalmic/optometry technicians operating medical instruments. Taking the Humphrey visual field analyzer as an example, the subject, under the guidance of a professional, performs visual field testing through the Humphrey visual field analyzer. The visual field analyzer will randomly display small light spots of thresholding intensity at different positions. If the subject sees the light spots, the subject will respond with a button accordingly as soon as possible, and then a testing result of the subject is obtained when all locations have been tested. The threshold values of each stimulus presented at that position is determined under a complex mathematic model (Swedish Interactive Threshold Algorithm). The testing result mainly includes mean deviation (MD) and pattern standard deviation (PSD) indicators.

However, due to the large size and high operational requirements of medical instruments such as Humphrey visual field analyzer, it is difficult especially for patients with limited mobility to go to places with such medical instruments for testing, and such medical instruments are not readily available for areas with limited medical resources, which limits the application of visual field testing. Thus, there is an urgent need for a rapid and simple method for visual field testing that can be applied to daily visual field testing or preliminary visual field inspection of subjects.

### SUMMARY

The present application provides a method and a device for visual field testing, which can conduct the visual field testing quickly and with simple operation.

In accordance with a first aspect, a method for visual field testing is provided, which is applied to a piece of display equipment, the display equipment includes a display, and the display is used to present a detection interface. The method includes steps of: displaying, within a preset first duration, a central stimulus point on a detection interface; displaying, within a preset second duration, a central target and a peripheral target on the detection interface based on a preset target display strategy, where the preset target display strategy is used to determine display data of the central target and the peripheral target; determining, in response to a subject's first trigger operation on the detection interface, a first response result corresponding to the first trigger operation and a target position area of a next round of display of the peripheral target; and determining a testing result of the subject based on the first response result corresponding to a preset number of detections, the testing result includes attended degree and attention area.

In the present application, the subject is guided to trigger operations by displaying the central stimulus point, the central target and the peripheral target on the display equipment respectively, and then the subject's response results to the central target and the peripheral target are obtained according to the subject's trigger operations, thereby the testing result of the subject's visual field can be quickly and simply obtained.

In a possible implementation, the method also includes steps of: determining a quality indicator area on the detection interface based on the attended degree; displaying the central target and displaying a judgment target in the quality indicator area based on the preset target display strategy within a preset third duration; determining, in response to the subject's second trigger operation on the detection interface, a second response result corresponding to the second trigger operation; and determining a quality indicator of the subject during testing based on the second response result and a preset number of indicator trials.

In a possible implementation, the method also includes steps of: indicating that the subject's testing result is inaccurate if a saccade, a missing of the central target, a false negative or a false positive is presented in the quality indicator; and indicating that the subject's testing result is accurate if the saccade, the missing of the central target, the false negative and the false positive are not presented in the quality indicator.

In a possible implementation, the quality indicator includes a saccade rate, a missing rate, a false positive rate and a false negative rate, and the method also includes that: if the saccade rate is higher than a preset first threshold, it is indicated that the saccade is detected during a testing process of the subject; if the missing rate is higher than a preset second threshold, it is indicated that the missing of the central target is detected during the testing process of the subject; if the false positive rate is higher than a preset third threshold, it is indicated that the false positive is detected during the testing process of the subject; and if the false negative rate is higher than a preset fourth threshold, it is indicated that the false negative is detected during the testing process of the subject.

The use of quality assurance mechanism in visual field testing helps to objectively quantify and verify the reliability and accuracy of testing result, and also adds rigor to the testing result.

In a possible implementation, the display data includes a stimulus content, a stimulus angular size and a display position, and the step of displaying the central target and the peripheral target based on the preset target display strategy further includes steps of: determining the stimulus content of the central target and the stimulus content of the peripheral target based on a random algorithm and a preset display set; determining the stimulus angular size of the central target and the stimulus angular size of the peripheral target based on a target adjustment rule; determining the display position of the peripheral target based on the target adjustment rule, the target position area and a preset detection range. The preset target display strategy includes the random algorithm and the target adjustment rule.

In a possible implementation, the method also includes steps of: dividing the detection interface based on a preset division threshold to obtain target meridians in the detection interface, where the target meridians include a first meridian and multiple second meridians; and displaying, within the preset second duration, the peripheral target on the first meridian.

In a possible implementation, the method also includes a step of: displaying, within the preset second duration, peripheral stimulus points on the multiple second meridians. The peripheral stimulus points and the peripheral target have the same eccentricity.

In the detection interface, by introducing meridians for limiting the position of the peripheral target, it is possible to achieve a limited number of detections, improve the accuracy of the testing, and improve the testing efficiency.

In a possible implementation, the method also includes steps of: obtaining initial information of the subject; determining a test report of the subject based on the initial information and the testing result, and storing data in the test report of the subject.

By storing the data in the test reports of the subject at different periods, the historical data of the subject can be formed to monitor the development of the subject's visual field or detect the therapeutic effect of the subject.

In a possible implementation, the method also includes steps of: determining age information of the subject based on the initial information; obtaining, based on the age information of the subject, age-matched visual field data corresponding to the age information, and determining comparison data of the testing result and the age-matched visual field data. The comparison data can be used to provide data support for evaluating the testing result of the user's visual field.

In accordance with a second aspect, a device for visual field testing is provided, which includes a display module, a response module and a processing module.

The display module is configured to display, within a preset first duration, a central stimulus point on a detection interface; and is also configured to display, within a preset second duration, the central target and the peripheral target on the detection interface based on a preset target display strategy. The preset target display strategy is used to determine display data of the central target and the peripheral target, and the preset second duration is less than the preset first duration.

The response module is configured to determine, in response to the subject's first trigger operation on the detection interface, a first response result corresponding to the first trigger operation and a target position area of a next round of display of the peripheral target.

The processing module is configured to determine the subject's testing result based on the first response results corresponding to a preset number of detections, and the testing result includes an attended degree and an attention area.

### DESCRIPTION OF THE DRAWINGS

FIG. 1 is a schematic structure diagram of a piece of display equipment in some embodiments of the present application;

FIG. 2 is a schematic flow diagram of a method for visual field testing provided by an embodiment of the present application;

FIG. 3 is a schematic diagram of a detection interface in some embodiments of the present application;

FIG. 4 is a schematic diagram of a detection interface in some embodiments of the present application;

FIG. 5 is a schematic diagram of the detection interface in some embodiments of the present application;

FIG. 6 is a schematic flow diagram another method for visual field testing provided by an embodiment of the present application;

FIG. 7 is a schematic diagram of the detection interface in some embodiments of the present application;

FIG. 8 is a schematic flow diagram of another method for visual field testing provided by an embodiment of the present application;

FIG. 9 is a schematic diagram of an attention area in comparative experimental testing results provided by an embodiment of the present application;

FIG. 10 is a schematic diagram of a detection interface in the method for visual field testing provided by an embodiment of the present application; and

FIG. 11 is a schematic diagram of a device for visual field testing provided by an embodiment of the present application.

### DETAILED DESCRIPTION OF THE EMBODIMENTS

The technical solutions in the embodiments of the present application will be introduced herein below in combination with the drawings in the embodiments of the present application. In the description of the embodiments of the present application, unless otherwise specified, the symbol “/” means or, for example, A/B may mean A or B; “and/or” in this article is only a description of the association relationship of the associated objects, indicating that there may be three relationships, for example, A and/or B may represent three scenarios: the sole existence of A, the coexistence of A and B, and the sole existence of B. In addition, in the description of the embodiments of the present application, the term “multiple” means two or more than two.

In the following, the terms “first”, “second”, and “third” are used only for descriptive purposes and cannot be understood as indicating or implying relative importance or implicitly indicating the number of technical features indicated. Therefore, the features defined as “first”, “second”, and “third” may explicitly or implicitly include one or more of the features.

For the purpose of explanation rather than limitation, specific details such as specific system structures and technologies are presented to facilitate a thorough understanding of the embodiments of the present application. However, it should be clear to persons skilled in the art that the present application may also be implemented in other embodiments without these specific details. In other cases, detailed descriptions of well-known systems, devices, circuits, and methods are omitted to avoid unnecessary details that hinder the description of the present application.

For ease of understanding, some concepts involved in the embodiments of the present application are introduced and explained below:

### (1) Visual Field

The visual field, also known as field of view, refers to the spatial range that can be seen when the head and eyeballs of a person are fixed and the eyes are looking at objects directly in front of the person, which is called static visual field, and the range seen by the eyes when the eyes are moving is called dynamic visual field which is usually expressed in angles. The size and shape of the visual field are related to the distribution of sensory cells on the retina. The range of the visual field may be measured by a perimeter.

### (2) Mean Deviation

The mean deviation (MD) is the main indicator in the testing result of the Humphrey visual field analyzer. MD reflects an average deviation of the subject's visual field from the normal visual field. The more MD deviates from zero, the greater the difference between the visual field and the normal visual field.

For example, in patients with glaucoma, as the disease progresses, the MD value gradually increases to a negative value, indicating a decrease in the overall visual sensitivity and the occurrence of local visual field defects.

### (3) Humphrey Visual Field Testing

The Humphrey visual field analyzer mainly evaluates the range and quality of the visual field by detecting a light sensitivity of different visual field areas. It uses the Swedish Interactive Threshold Algorithm (SITA) to dynamically adjust the brightness of the test point according to the subject's response to the test and find a sensitivity threshold of the retina to light stimulation. During the examination, the subject needs to fix his/her gaze on a fixed point ahead, and the visual field analyzer will randomly flash light spots of different brightness around the fixed point. After the subject sees the light spots, he/she should press the button to obtain the range and sensitivity of the subject's visual field.

### (4) Stimulus Point

The stimulus point is the position of a specific visual stimulus presented to the subject during a visual field testing. Usually, such stimulus points are distributed in different areas of the visual field in a certain pattern. The stimulus point may be presented in different forms, the most commonly-presented form is a light spot. The angular size, brightness and duration of the light spot may be adjusted according to the needs of detection. In addition, the stimulus point may also be a graphic, a line or other visual elements.

It should be understood that the visual field testing is usually performed by professional doctors and ophthalmic technicians operating medical instruments. Taking the Humphrey visual field analyzer as an example, the Humphrey visual field analyzer has the characteristics of large size and high operational requirements. In particular, it is difficult for patients with mobility issues to go to places equipped with such medical instruments for detection. Moreover, for areas with limited medical resources, such medical instruments are difficult to obtain at any time, which limits the application of visual field testing.

In view of this, a method and a device for visual field testing are provided in the embodiments of the present application. The method is applied to display equipment, and the display equipment includes a display for presenting a detection interface. The method includes steps of: displaying, within a preset first duration, a central stimulus point on the detection interface; displaying, within a preset second duration, a central target and a peripheral target on the detection interface based on a preset target display strategy; and determining, in response to a subject's first trigger operation on the detection interface, a first response result corresponding to the first trigger operation and a target position area of the peripheral target displayed in a next round; and then determining a testing result of the subject based on the first response results corresponding to a preset number of detections. In the present application, the subject is guided to trigger operations by displaying the central stimulus point, the central target and the peripheral target on the display equipment respectively, and then the subject's response results to the group of targets (central target and peripheral target) are obtained according to the subject's trigger operations, thereby the testing result of the subject's visual field is obtained quickly and simply.

The method for visual field testing provided in the embodiment of the present application may be applied to the display equipment such as a tablet computer, a desktop computer, a laptop computer, a smart TV, etc. The embodiment of the present application does not impose any restrictions on the specific type of the display equipment.

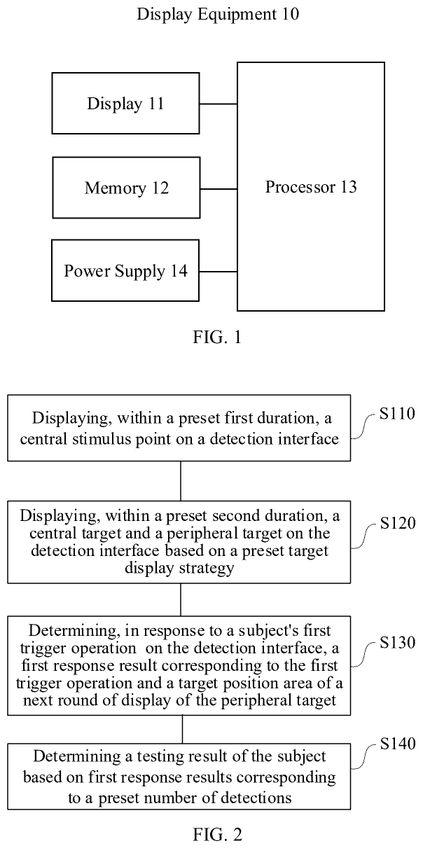

For example, FIG. 1 is a schematic structure diagram of the display equipment in some embodiments of the present application. As shown in FIG. 1, the display equipment 10 includes a display 11, a memory 12, a processor 13, and a power supply 14.

The display equipment 10 includes one or more processors 13, and the one or more processors 13 may support the display equipment 10 to implement the method for visual field testing in the method embodiment. The processor 13 may be a general processor 13 or a dedicated processor 13. For example, the processor 13 may be a central processing unit 13 (CPU), a digital signal processor 13 (DSP), an audio processor 13, a graphics processor 13, an application specific integrated circuit (ASIC), a field programmable gate array (FPGA) or other programmable logic devices.

The processor 13 may be used to control the display equipment 10, execute software programs, and process data of the software programs. The display equipment 10 may also include a communication unit to realize the input (reception) and output (transmission) of signals.

The display 11 has a detection interface configured for displaying images and videos, for example, for visual field testing.

The display equipment 10 may include one or more memories 12, on which programs are stored, and the programs may be run by the processor 13 to generate instructions, to cause the processor 13 to execute the method for visual field testing described in the embodiments according to the instructions.

The memory 12 may also store data. The processor 13 may also read the data stored in the memory 12, the data may be stored at the same storage address as the program, or the data may be stored at a different storage address from the program.

The processor 13 and the memory 12 may be set up separately or integrated together. For example, the processor 13 and the memory 12 are integrated on a system on chip (SOC) of terminal equipment.

The display equipment 10 may also include a mobile communication module, a wireless communication module, an audio module, a speaker, etc.

The wireless communication module may provide solutions for wireless communication applications on the display equipment 10, including wireless local area networks (WLAN) (such as wireless fidelity (Wi-Fi) networks), Bluetooth (BT), global navigation satellite system (GNSS), frequency modulation (FM), near field communication (NFC), etc. The wireless communication module may be one or more devices integrating at least one communication processing module. The wireless communication module receives electromagnetic waves via an antenna, frequency-modulates and filters the electromagnetic wave signals, and sends the processed signals to the processor. The wireless communication module may also receive the signals to be sent from the processor, frequency-modulate the signals, amplify the signals, and then convert the signals into electromagnetic waves through the antenna for transmission.

For ease of understanding, the following embodiments of the present application will take the display equipment having the structure shown in FIG. 1 as an example, the detection interface is displayed on the display of the display equipment, and the method for visual field testing provided by the embodiment of the present application is described in detail in combination with FIG. 2 to FIG. 10 and the application scenarios.

FIG. 2 is a flow chart of a method for visual field testing provided by an embodiment of the present application. As shown in FIG. 2, the method includes the following steps S110 to S140:

In step S110, a central stimulus point is displayed on a detection interface within a preset first duration.

Herein, the detection interface is mainly used to define a display area for visual field testing. The detection interface is used to limit a display range of the peripheral stimulus points in the visual field testing, and determine the display position of the central stimulus point, thereby facilitating the determination of display positions of the peripheral stimulus points in each round of detection.

It should be understood that under normal circumstances, the visual field range of a person's monocular (left eye or right eye) is roughly elliptical. It should be noted that the visual field range of each person may vary due to individual differences, age and other factors.

It should be understood that the display area for visual field testing in a display interface is greater than or equal to the maximum visual field range of a monocular eye. The display area may be a square area or a circular area, etc. The present application does not impose any restrictions on the type of display area.

In some embodiments, the detection interface also includes an information area for presenting information. The information area may be provided at an edge or corner of the detection interface and may be used to display relevant information during a testing process. The information area may also be used to display initial information of the subject. The information area may also display parameter settings of this test.

For example, displaying the progress of the test that has been completed for the current ongoing and the patient's response to the stimulus point (such as the number of correct responses, the number of incorrect responses, the number of non-responses, etc.), displaying the subject's code, name, date of birth, test date, etc.; and displaying an intensity of the stimulus point, the preset first duration, the preset second duration, a response duration, etc.

It should be understood that the central stimulus point is a fixation point located in the center of the display interface and is an obvious marker. The central stimulus point may be a cross, a circle or other graphics to help the subject focus on the central stimulus point as much as possible during the entire process of visual field testing to ensure the accuracy of the testing result, this is because the gaze position of the eyeball will directly affect the results of the visual field testing.

In some embodiments, before step S110, the method also includes a step of obtaining the initial information of the subject, and the initial information includes the subject's name, code, gender, date of birth, the eye to be tested, and the maximum degree expected to be detected.

Herein, the code is used to store test data of the subject, and the test data includes the initial information and the testing result. The date of birth is used to determine the age information of the subject; the eye to be tested is used to distinguish whether the eye to be tested is the left eye, the right eye or both eyes. The maximum degree expected to be detected is used to determine a preset detection range of this test.

It should be understood that for subjects in different situations, the corresponding maximum degree of testing is different. For example, for subjects who have never undergone visual field testing, the corresponding maximum degree of testing is the maximum value in the visual field testing, and the corresponding preset detection range is also the maximum value. For subjects who have been diagnosed with glaucoma, if the previous maximum degree of visual field testing is 24°, then the corresponding maximum degree of testing is greater than 24°, which may be set to 30°, and the corresponding preset detection range is 30°.

It should be understood that different maximum degrees expected to be detected correspond to different preset detection ranges, and thus, the detection accuracy is also different. In this way, for different subjects the detection accuracy of the subject may be improved optionally by setting the maximum degree expected to be detected.

It should also be understood that the central stimulus point is displayed for the preset first duration, which facilitates the subject to better focus on the stimulus point, reduce the deviation of the line of sight, and improve the accuracy of the testing. Herein, the preset first duration may be 800 milliseconds (ms), or 900 ms, etc.

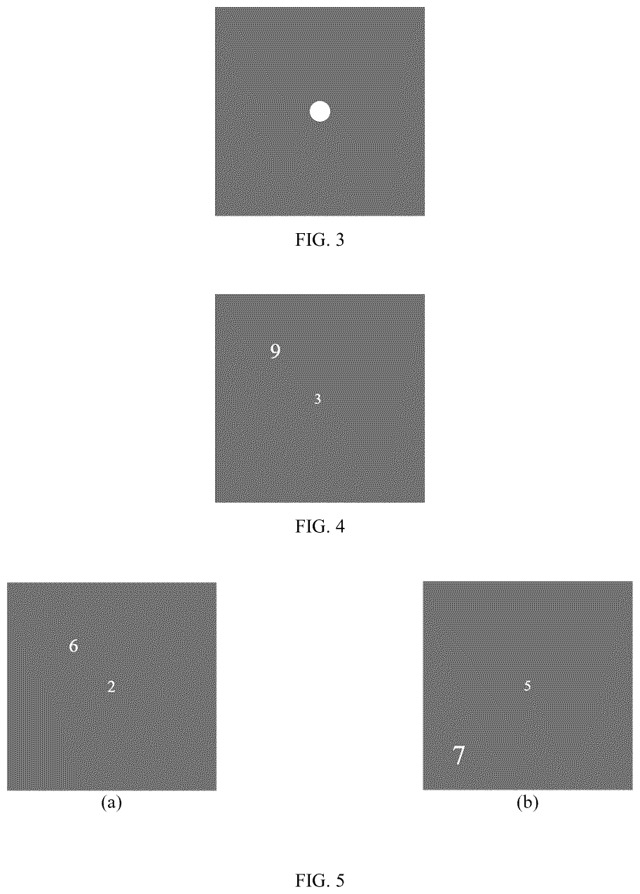

FIG. 3 is a schematic diagram of the detection interface in some embodiments of the present application. As shown in FIG. 3, the detection interface has a square display area, and a circular stimulus point, that is, the central stimulus point, is displayed at the center of the display area. The display time of the central stimulus point is the preset first duration.

In some embodiments, the angular size of the central stimulus point may also be limited. It should be noted that the testing result will be affected if the angular size of the central stimulus point is too large or too small. Therefore, it is necessary to configure the angular size of the central stimulus point within the appropriate range for the subject's fixation, as to as reduce the deviation of the line of sight and facilitate the improvement of the accuracy of the testing. For example, the circular stimulus in FIG. 3 has 0.5° angular size. It should be understood that the angular size of the circular stimulus is also related to the distance between the subject and the display in the display equipment.

In step S120, a central target and a peripheral target are displayed in the detection interface based on a preset target display strategy within a preset second duration.

The preset second duration is a duration for displaying the central target and the peripheral target. The display of the central target and the peripheral target is used to judge a sensitivity of the subject to the peripheral target. Thus, the preset second duration is often relatively short. In some embodiments, the preset second duration is less than the preset first duration, which improves the detection accuracy. For example, the preset first duration is 800 ms and the preset second duration is 250 ms.

It should be understood that the central target is used to detect whether the subject maintains fixation on the central position, and the peripheral target is used to detect the subject's attention area (AA) and attended degree (AD). Through comparative experiments, it can be determined that AA and AD are accurate in judging the result of visual field testing.

Herein, the attention area may also be called the visual attention area (VAA), that is, the maximum range of visual field that can be attended to when the individual subject is focused on the central target.

The attended degree refers to a range of the stimulus area that an individual subject can simultaneously focus on during a visual attention process. Specifically, AD is used to measure the breadth of attention to the surrounding peripheral target stimuli while paying attention to the central target stimulus.

Herein, the preset target display strategy is used to determine the display data of the central target and the peripheral target, and the display data includes a stimulus content, a stimulus angular size and a display position (i.e., spatial positioning).

It should be understood that the central target and the central stimulus point have the same display position but different stimulus contents, and the type of content displayed by the central target and the peripheral target may be the same or different.

Herein, the central target or the peripheral target may be one of numbers, letters, patterns, Chinese characters, or geometric shapes, that is, the central target and the peripheral target may both be numbers. It may also be that the central target is a number and the peripheral target is a letter. It may also be that the central target is a letter and the peripheral target is a pattern, etc. For example, the central target is the number 3 and the peripheral target is the number 9. Or alternatively, the central target is the number 3 and the peripheral target is the letter A. Or alternatively, the central target is the number Z and the peripheral target is a turtle pattern, etc.

In general, the shapes of the central target and the peripheral target are different from the shape of the central stimulus point, which is convenient for the subject to distinguish the target and the stimulus point, and to identify the target. Herein, the target refers to the central target and the peripheral target, and the stimulus point refers to the central stimulus point and the peripheral stimulus point as mentioned below.

It should be noted that the central target and the peripheral target may also have the same stimulus content. For example, the central target and the peripheral target are both the number 3.

FIG. 4 is a schematic diagram of the detection interface in some embodiments of the present application. As shown in FIG. 4, the detection interface has a square display area, and the central target 3 is displayed at the center of the display area, and the peripheral target 9 is displayed around the central position.

For the determination of the stimulus angular size of the central target or the peripheral target, it may be that both the central target or the peripheral target are configured having a fixed angular size based on the preset target display strategy. Or alternatively, the central target may be configured having a fixed angular size, while the angular size of peripheral target may be adjusted according to the display position. For example, when the display position of the peripheral target is close to the central target, the stimulus angular size of the peripheral target becomes smaller; when the display position of the peripheral target is far from the central target, the stimulus angular size of the peripheral target becomes larger.

In some embodiments, the stimulus angular size may be calculated by the following formula: stimulus angular size=0.4*(1+eccentricity/4).

FIG. 5 is a schematic diagram of the detection interface in some embodiments of the present application. As shown in FIG. 5, the peripheral target being displayed at a position close to the central target is presented in pattern (a), where the central target 2 and the peripheral target 6 are displayed in the display area. The peripheral target being displayed at a position far from the central target is presented in pattern (b), where the central target 5 and the peripheral target 7 are displayed in the display area. The stimulus angular size of the peripheral target 7 is larger than the stimulus angular size of the peripheral target 6, and the stimulus angular size of the central target 5 is the same as the stimulus angular size of the central target 2.

In some embodiments, the display data includes a stimulus content, a stimulus angular size and a display position (i.e., spatial positioning), and the preset target display strategy includes a random algorithm and a target adjustment rule. The step S120 of displaying the central target and the peripheral target based on the preset target display strategy includes the following:

Firstly, the stimulus content of the central target and the stimulus content of the peripheral target are determined based on the random algorithm and a preset display set.

The preset display set refers to a set of stimulus contents that may be used for the central target and the peripheral target, and the preset display set may be one or more of numbers, letters, patterns, Chinese characters, or geometric figures. The stimulus content of the central target and the stimulus content of the peripheral target are selected from the preset display set through a random algorithm.

For example, it may be determined through the random algorithm that the central target is A and the peripheral target is 3 if the preset display set includes: 1, 2, 3, 4, 5, 6, 7, 8, 9, A, B, C, D, E, F and G. It may be determined through the random algorithm that the central target is 8 and the peripheral target is 2 if the preset display set includes: 1, 2, 3, 4, 5, 6, 7, 8, 9 and 0.

It should be understood that if both the stimulus content of the central target and the stimulus content of the peripheral target are set as numbers, and these numbers can meet the display requirements, then the corresponding stimulus content may be directly determined through a random algorithm.

It should also be understood that the above random algorithm may be a function for determining random numbers in a programming language, or a shuffling algorithm, etc., and the embodiment of the present application does not impose any restrictions on the random algorithm.

Secondly, the stimulus angular size of the central target and the stimulus angular size of the peripheral target are determined based on the target adjustment rule.

Herein, the target adjustment rule is applied to adjust the stimulus angular size of the central target and the peripheral target. The central target and the peripheral target may be configured having a fixed angular size. Or alternatively, the central target may be configured having a fixed angular size, while the angular size of the peripheral target may be adjusted according to the display position. For example, when the display position of the peripheral target is close to the central target, the stimulus angular size of the peripheral target becomes smaller; when the display position of the peripheral target is far away from the central target, the stimulus angular size of the peripheral target becomes larger.

Finally, the display position of the peripheral target is determined based on the target adjustment rule, the target position area and the preset detection range.

It should be understood that the display position of the central target remains unchanged. By adjusting the display position of the peripheral target, the subject's reaction to the peripheral targets in different directions is detected to determine the subject's visual sensitivity in various directions.

Herein, the target position area is the main basis for adjusting the angular size of the peripheral target. The target position area is a dynamically adjusted area, and the target position area of this round will be determined according to the maximum degree expected to be detected and the first response result obtained in the previous round of detection.

In some embodiments, the display position of the peripheral target may be determined through parameter estimation by sequential testing (PEST). It should be understood that the display position of the peripheral target may be determined by eccentricity.

For the first round, the display positions of the central target and the peripheral target may be determined by the maximum degree expected to be detected, and for the second round and subsequent rounds, the display positions of the central target and the peripheral target may be determined by the first response result from their previous round.

The display position of the peripheral target is dynamically adjusted according to the target adjustment rule, the target position area and the preset detection range. The maximum degree expected to be detected is used to determine the preset detection range of this detection.

In step S130, a first response result corresponding to a first trigger operation and a target position area of a next round of display of the peripheral target are determined in response to the subject's first trigger operation on the detection interface.

It should be understood that, at this time, the detection interface displays a trigger area. The trigger area is used to interact with the subject, that is, a group of options including the stimulus content of the central target and the peripheral target are displayed in the trigger area, and in response to the subject's first trigger operation on the detection interface, the central target and the peripheral target selected by the subject can be determined.

For example, in one detection, if the central target and the peripheral target are as shown in FIG. 4, the group of options displayed in the trigger area may be 1, 2, 3, 4, 5, 6, 7, 8, 9, 0, in which the central target 3 and the peripheral target 9 are included.

It can be seen from the application scenario that for most subjects of visual field testing, there are more or less missing visual fields. The trigger area may be presented in the center of the detection interface to facilitate the subject to intuitively perform a trigger operation, that is, the first trigger operation.

Before entering the detection interface for performing the method for visual field testing, the subject may be helped to understand the usage method through video or text instructions, including the trigger operation for the trigger area, for example, the text instruction “Click on what you see, first select the content seen in the middle, and then select the content seen around”.

The corresponding first response result is determined by the display equipment according to the subject's first trigger operation (including two triggers of the central target and the peripheral target). It should be understood that the subject will trigger twice. If both triggers are correct, the corresponding first response result is a correct response. If there is one trigger error or two trigger errors, the corresponding first response result is an incorrect response.

The target position area of the next round of display of the peripheral target may also be determined by the display equipment according to the subject's first trigger operation. It should be understood that if the first response result is a correct response, it is indicated that the subject can see the peripheral targets at the same time as the central target is being focused on, in this case, the display range of the peripheral target needs to be further expanded. If the first response result is an incorrect response, it means that the subject cannot see the peripheral targets at this moment, or the subject's attention has deviated. At this point, the display range of the peripheral targets should be reduced, that is, the target position area of the next round of display of the peripheral target should be adjusted.

In step S140, a testing result of the subject is determined based on first response results corresponding to a preset number of detections.

It should be understood that steps S110 to S130 correspond to the first response result obtained in one testing process. The embodiment of the present application needs to determine the testing result of the subject according to the first response results corresponding to the preset number of detections.

Herein, the testing result includes an attended degree and an attention area, and the visual field of the subject is presented through the attended degree and the attention area.

The method for visual field testing provided in the embodiment of the present application is applied to display equipment, and the display equipment includes a display for presenting a detection interface. The central stimulus point is displayed on the detection interface within the preset first duration. The central target and the peripheral target are displayed on the detection interface based on the preset target display strategy within the preset second duration. In response to the subject's first trigger operation on the detection interface, the first response result corresponding to the first trigger operation and the target position area of the peripheral target displayed in the next round are determined. Then, the testing result of the subject is determined based on the first response results corresponding to the preset number of detections. In the present application, the subject is guided to trigger operations by displaying the central stimulus point, the central target and the peripheral target on the display equipment respectively, and then the subject's response results to the central target and the peripheral target are obtained according to the subject's trigger operations, thereby the testing result of the subject's visual field is obtained quickly and simply.

It should be understood that the method for visual field testing provided in the embodiment of the present application is different from a dedicated system based on professional hardware (such as the Humphrey visual field analyzer). The method for visual field testing here may be deployed on a piece of display equipment through an application, a web page, etc., or may be a dedicated software system for use with the display equipment, which enhances the application scope and adaptability of visual field testing, and makes the method more accessible for application in various scenarios.

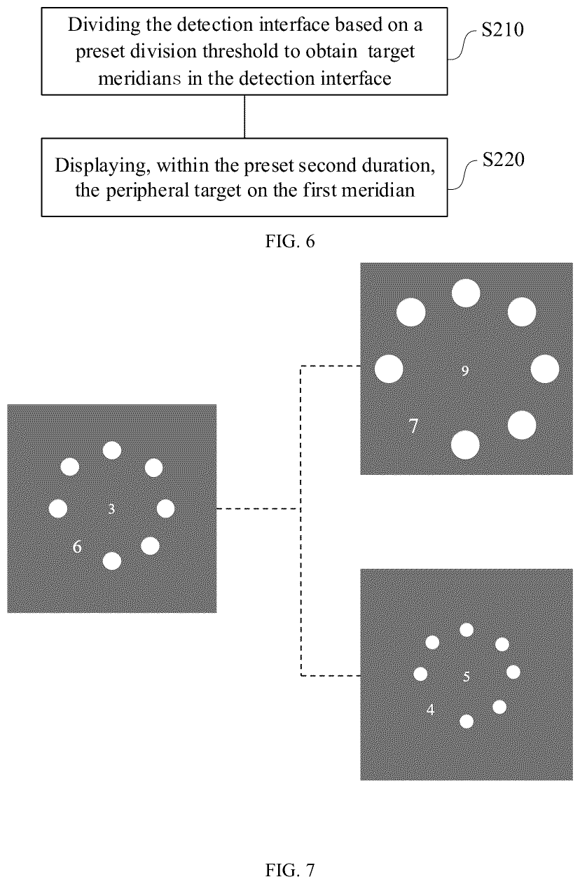

In some embodiments, in the detection interface, by introducing a meridian for limiting the position of the peripheral target, it is possible to improve the accuracy of testing and the testing efficiency through a limited number of detections. FIG. 6 is a flow diagram of another method for visual field testing provided in an embodiment of the present application. As shown in FIG. 6, the method also includes the following steps S210-S220:

In step S210, the detection interface is divided based on a preset division threshold to obtain target meridians in the detection interface.

The detection interface is divided by the target meridians, that is, taking the center point of the detection interface as the center of the circle, the target meridians correspond to multiple line segments extending from the center point to the edge of the detection interface. The multiple meridians determined in the detection interface for displaying peripheral targets are the target meridians.

It should be understood that the number of target meridians meets a preset division threshold. To allow the target meridians to cover the detection interface, the preset division threshold is greater than or equal to 3. For example, the number of target meridians is 4, 5, 6, 8, 10, 16, etc.

For example, if the preset division threshold is 3, the corresponding target meridians correspond to line segments in the directions of 0°, 120°, and 240° in the detection interface. If the preset division threshold is 5, the corresponding target meridians correspond to line segments in the directions of 0°, 72°, 144°, 216°, and 288° in the detection interface. If the preset division threshold is 8, the corresponding target meridians correspond to line segments in the directions of 0°, 45°, 90°, 135°, 180°, 225°, 270°, and 315° in the detection interface.

It should be understood that for the direction corresponding to each target meridian, multiple detections are required to obtain the attended degree in the corresponding direction. Thus, the number of target meridians can be reasonably determined through the preset division threshold, to improve the efficiency and accuracy of the testing.

It should also be understood that the method for visual field testing provided in the embodiment of the present application is different from professional equipment such as the Humphrey visual field analyzer, which obtains high-precision testing results through professional medical equipment. The method for visual field testing provided in the embodiment of the present application is a fast and simple detecting method, which can be deployed on tablets, computers and other commonly-used daily display devices, so that the testing result of the subject can be obtained quickly and conveniently at a high accuracy. Therefore, although the more target meridians there are, the more detailed the division of the detection interface is, and the more accurate the corresponding testing result is, for the embodiment of the present application, by reasonably setting the preset division threshold, the testing efficiency can be improved under the premise of meeting a certain testing accuracy. For example, the preset division threshold is 6 or 8.

Herein, the target meridians include a first meridian and a second meridian. The first meridian is used for configuring the peripheral target, and the second meridian is used for configuring the peripheral stimulus point. It should be understood that for each detection, only one peripheral target is displayed within the preset second duration. Hence, the first meridian is one of the multiple target meridians, and the other target meridians are all second meridians.

It should also be understood that step S210 may be a process prior to step S110 in FIG. 2, or may be a process prior to step S120 in FIG. 2.

In step S220, the peripheral target is displayed on the first meridian within the preset second duration.

The display direction of the peripheral target is limited by each first meridian in the target meridians. It should be understood that for each first meridian, the corresponding peripheral target will be displayed, and the target position area of peripheral target in the next round on the first meridian will be adjusted by the first response result of the subject.

For each first meridian, the sensitivity of the subject in the corresponding direction may be determined by limiting the number of displays of peripheral targets on the first meridian. For example, the number of displays of peripheral targets on each first meridian may be determined according to the preset number of detections. For example, if the preset number of detections is 48 times and the number of first meridians is 8, then 6 detections are required for each first meridian.

In some embodiments, the subject's visual field perception can be improved by displaying peripheral stimulus points on the second meridians, and thus the detection accuracy can be improved. Hereby, the method for visual field testing also includes a step of: displaying the peripheral target on the first meridian and the peripheral stimulus point on the second meridians within the preset second duration, where the peripheral stimulus points and the peripheral target have the same eccentricity.

Herein, the peripheral stimulus points may also be used as fixation references. To view the central target and the peripheral target, the subject may have a visual field that deviates from the central position. By setting the peripheral stimulus points, the accuracy of the result for visual field testing can be improved.

It should be understood that, in the initial setting of the peripheral target, the peripheral target may be set at a central position on the first meridian corresponding to the maximum degree expected to be detected. If the subject's first response result to the peripheral target and the central target is a correct response, the next peripheral target is set on the first meridian corresponding to a region from the central position of the first meridian to the edge position of the detection interface. If the subject's first response result to the peripheral target and the central target is an incorrect response, the next peripheral target is set on the first meridian corresponding to a region from the central position of the first meridian to the center point of the detection interface. In addition, the subsequent peripheral target may also be set at a new central position in the corresponding region. In other words, the display positions of the subsequent peripheral targets are determined through the strategy of gradual approach on the first meridian, thereby achieving comprehensive detections on the first meridian.

For example, the preset division threshold is 8, and the eight target meridians correspond to line segments in the directions of 0°, 45°, 90°, 135°, 180°, 225°, 270°, and 315° in the detection interface, respectively. The first meridian may correspond to one of the directions of 0°, 45°, 90°, 135°, 180°, 225°, 270°, and 315°. For example, if the first meridian corresponds to 225°, then the second meridians refer to 0°, 45°, 90°, 135°, 180°, 270°, and 315°. FIG. 7 is a schematic diagram of the detection interface in some embodiments of the present application. As shown in FIG. 7, pattern (a) of FIG. 7 shows that, during a detection where the first meridian corresponds to 225°, the central target 3, the peripheral target 6, and the peripheral stimulus points on the second meridians are displayed in the detection interface within the preset second duration in response to the subject's first trigger operation on the detection interface.

If the first response result corresponding to the first trigger operation is indicated as a correct response, in the next detection, pattern (b) of FIG. 7 is displayed within the preset second duration, the central target 9 is displayed on the detection interface, the peripheral target 7 is displayed as extending outward on the first meridian, and the peripheral stimulus points are displayed at the corresponding position on the second meridians, respectively. It should be noted that the peripheral target and peripheral stimulus points in pattern (b) of FIG. 7 are greater in angular size than the peripheral target and peripheral stimulus points in pattern (a) of FIG. 7.

If the first response result corresponding to the first trigger operation is indicated as an incorrect response, in the next detection, pattern (c) of FIG. 7 is displayed within the preset second duration, the central target 5 is displayed on the detection interface, the peripheral target 4 is displayed as extending inward on the first meridian, and the peripheral stimulus points are displayed at the corresponding position on the second meridians, respectively. It should be noted that the peripheral targets and peripheral stimulus points in pattern (c) of FIG. 7 are smaller in angular size than the peripheral target and peripheral stimulus points in pattern (a) of FIG. 7.

Through the embodiment in FIG. 7, it can also be understood that the central target and the peripheral target are different for different detection iterations. By using random algorithms, different central targets and peripheral targets are displayed in different detection iterations. This random approach enhances the accuracy of detections.

It should also be understood that it is also possible to start the detections from the position where the first meridian is connected to the central target and gradually toward the edge of the maximum degree expected to be detected. Or alternatively, the detections may be started from the edge of the maximum degree of the preset inspection and along the first meridian, and gradually towards the position where the first meridian is connected to the central target. The first response results at different positions of the first meridian are recorded.

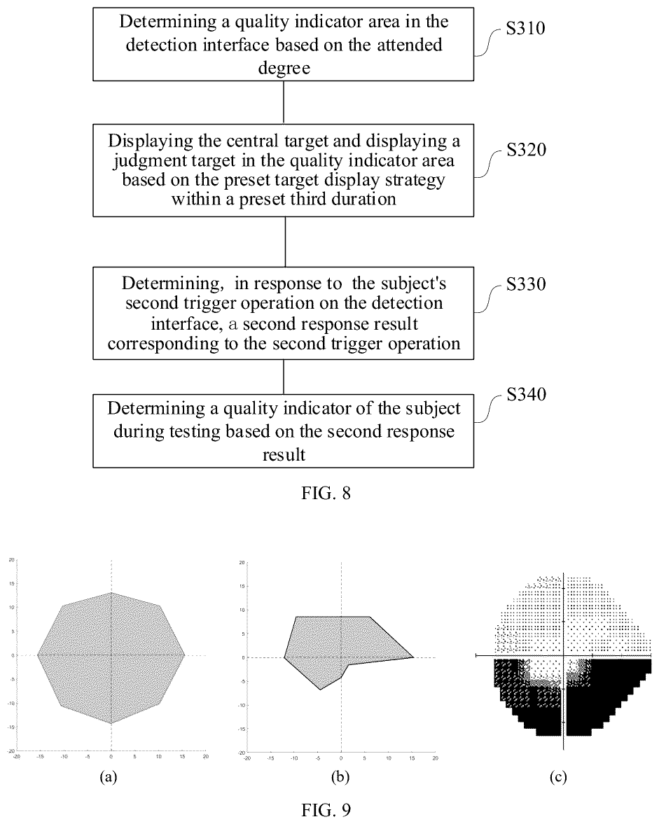

In some embodiments, the testing result obtained by the above method is combined with a quality assurance mechanism to improve the reliability and validity of the visual field detection result. FIG. 8 is a flow diagram of another method for visual field testing provided by an embodiment of the present application, which is used to provide the quality assurance mechanism. As shown in FIG. 8, the method further includes the following steps S310-S340 under the content shown in FIG. 2:

In step S310, a quality indicator area in the detection interface is determined based on the attended degree.

The testing result is obtained after the preset number of detections are completed, and the testing result includes the attended degree and the attention area. Then, based on each attended degree, the quality indicator area is determined by a preset range. For example, the preset range may refer to a range corresponding to the attended degree within ±3°, which leads to the determination of the quality indicator area; the preset range may also refer to a range corresponding to the attended degree within ±5°.

It should be understood that for the detection interface having the target meridians, the quality indicator area may be determined according to the attended degree corresponding to each target meridian. In other words, the quality indicator area is determined according to a preset range based on the attended degree corresponding to each target meridian.

In some embodiments, the quality indicator area may also be determined in the detection interface based on the attention area, and the quality indicator area is determined according to the preset range based on the boundary of the attention area.

In step S320, within a preset third duration, based on the preset target display strategy, the central target is displayed and a judgment target is displayed in the quality indicator area.

Herein, prior to step S320, it is also required to display, within the preset first duration, the central stimulus point in the detection interface, that is, the quality assurance mechanism is the same as the testing process.

It should be understood that the preset third duration may be equal to the preset second duration, or may be different from the preset second duration.

The display data of the judgment target is similar to that of the peripheral target, except that the display positions of the two are different, and the judgment target needs to be displayed in the quality indicator area.

The judgment targets on each first meridian may be configured to be displayed both within and outside the attended degree in the quality indicator areas, thereby the accuracy of the quality assurance mechanism is enhanced.

In step S330, a second response result corresponding to the second trigger operation is determined in response to the subject's second trigger operation on the detection interface.

It should be understood that the second trigger operation is the same as the first trigger operation, references may be made to the description of step S130 above.

In step S340, a quality indicator of the subject during testing is determined based on a preset number of indicator trials and the second response result.

Herein, the preset number of indicator trials is used to limit the number of times the quality assurance mechanism is operated. For instance, when there are 8 target meridians, there will be one occurrence each for both the internal and external attended degree, totaling 16 times. Thus, the preset number of indicator trials is set to 16 times.

If a saccade, a missing of the central target, a false negative or a false positive is presented in the quality indicator, it is indicated that the testing result of the subject is inaccurate. If the saccade, the missing of the central target, the false negative and the false positive are not presented in the quality indicator, it is indicated that the testing result of the subject is accurate.

Herein, the quality indicators include a saccade rate, a missing rate, a false positive rate and a false negative rate. The saccade rate represents the proportion of the number of responses made by the subject that are correct for peripheral targets but incorrect for central targets to the total number of the preset number of indicator trials. When this indicator is high, it is indicated that the subject's eyes have been jerking frequently and has failed to maintain central fixation as required by the task.

The missing rate indicates the proportion of the number of responses made by the subjects that are incorrect for central targets to the total preset indicator trials. When this indicator is high, it is indicated that the subjects' performance was poor.

False positive rate indicates the proportion of correct choices made by the subject when the detection stimulus appears outside the attended degree. For example, this value should range from 0/8 to 8/8, and the lower the better.

False negative rate indicates the proportion of incorrect choices made by the subject when the detection stimulus appears within the attended degree. For example, this value should range from 0/8 to 8/8, and the lower the better.

If the saccade rate is higher than a preset first threshold, it is indicated that there is a saccade during a testing process of the subject. If the missing rate is higher than a preset second threshold, it is indicated that there is a missing of the central target during the testing process of the subject. If the false positive rate is higher than a preset third threshold, it is indicated that there is a false positive during the testing process of the subject. If the false negative rate is higher than a preset fourth threshold, it is indicated that there is a false negative during the testing process of the subject.

It should be understood that other quality indicators may be provided for determining whether the testing result of the subject is accurate.

In the above embodiment, the initial information of the subject will be obtained. If the testing result is determined to be accurate through the quality assurance mechanism, a test report of the subject is determined based on the initial information and the testing result, and data in the test report of the subject is stored.

By storing the data of the test reports of the subject at different periods, the historical data of the subject can be formed, to monitor the development of the subject's visual field or test the therapeutic effect of the subject.

In some embodiments, age information of the subject may be determined by the date of birth in the initial information; then based on the age information of the subject, age-matched visual field data corresponding to the age information is obtained and comparison data of the testing result and the age-matched visual field data is determined. The comparison data may be used to provide data support for evaluating the testing result of visual field of the user.

For example, the comparative experiment involved M subjects from a first group who suffered from different types of visual field loss (including glaucoma patients, stroke patients, optic nerve damage patients, etc.) and M healthy subjects from a second group. It should be understood that the ages of these two groups of subjects were similar.

For these two groups of subjects, both were tested using the Humphrey visual field analyzer and the display equipment corresponding to the method for visual field testing provided in the embodiments of the present application, and the attended degree and attention area corresponding to each target meridian were obtained.

The mean values of the corresponding AD in the two groups of subjects in the comparative experiment were shown in table 1 below.

| | Target meridians | | | | | | | |
|---|---|---|---|---|---|---|---|---|
| | 0° | 45° | 90° | 135° | 180° | 225° | 270° | 315° |
| First group of | 10.8 | 9.5 | 7.3 | 9.7 | 11.6 | 12.2 | 9.8 | 9.9 |
| subjects | | | | | | | | |
| Second group | 15.6 | 14.6 | 13.1 | 14.6 | 15.6 | 15.0 | 14.4 | 14.4 |
| of subjects | | | | | | | | |

It should be understood that the statistical value of the data in table 1 is less than 0.01.

FIG. 9 is a schematic diagram of the attention area in testing results of the comparative experimental provided in the embodiment of the present application. As shown in FIG. 9, graphic (a) is a schematic diagram of the attention area of the second group of subjects, graphic (b) is a schematic diagram of the attention area of the first group of subjects, and graphic (c) shows the average deviation determined by the Humphrey visual field analyzer for the first group of subjects.

FIG. 9 shows that there is a strong correlation (positive correlation) between the attention area obtained by the method for visual field testing provided in the embodiment of the present application and the average deviation in the Humphrey visual field analyzer, indicating that the more serious the average deviation of the first group of subjects, the smaller the corresponding attention area.

Furthermore, through multiple comparative experiments, the correlation coefficient between the two was found to be 0.8, and a significance level is less than 0.001.

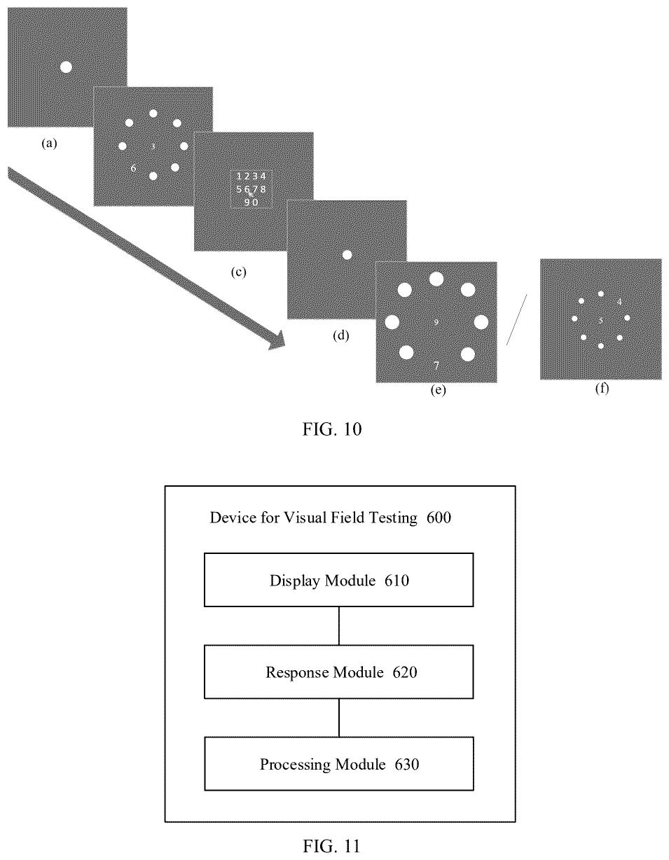

FIG. 10 is a schematic diagram of a detection interface in the method for visual field testing provided by the embodiment of the present application. As shown in FIG. 10, during a testing process, first, the central stimulus point in pattern (a) is displayed for 800 ms, then the central target, peripheral target and peripheral stimulus points in pattern (b) are displayed for 250 ms, and a group of options including the stimulus content of the central target and the peripheral target are displayed in a trigger area shown in pattern (c). The subject triggers the options corresponding to the central target and the peripheral target in turn by touching the screen with a mouse or a finger, and the response result corresponding to the trigger is obtained. If the response result is a correct response, in the next detection, the central target, the peripheral target and the peripheral stimulus points in pattern (e) are displayed for 250 ms, and if the response result is an incorrect response, in the next detection, the central target, the peripheral target and the peripheral stimulus points in pattern (f) are displayed for 250 ms.

Herein, in the subsequent detections, the central stimulus point in pattern (d) will also be displayed for 800 ms.

The embodiment of the present application provides a more sensitive and comprehensive testing for the visual ability of the subject. This method utilizes high-order visual processing and attention mechanisms to perform a richer test of the subject's actual visual field performance.

Meanwhile, the ability to automatically and self-pacedly perform visual field testing eliminates the interference from subjective human judgment and intervention, and reduces potential deviations and labor costs associated with manual testing.

It should be understood that the above are examples illustrating the application scenarios, and do not impose any limitations on the application scenarios of the present application.

It should be understood that the above examples are provided merely for the purpose of helping persons skilled in the art understand the embodiments of the present application, and are not intended to limit the embodiments of the present application to the specific numerical values or specific scenarios exemplified. Obviously, persons skilled in the art can make various equivalent modifications or changes based on the above examples given, and such modifications or changes also fall within the scope of the embodiments of the present application.

The method for visual field testing of the embodiment of the present application is described in detail in conjunction with FIGS. 2 to 10 above, and the device embodiment of the present application will be described in detail in conjunction with FIG. 11 below. It should be understood that the device for visual field testing in the embodiment of the present application may perform the various methods for visual field testing of the aforementioned embodiments of the present application, that is, for the specific working process of the following various products, references may be made to the corresponding process in the aforementioned method embodiments.

FIG. 11 is a schematic diagram of a device for visual field testing provided in an embodiment of the present application. It should be understood that the method for visual field testing shown in FIGS. 2 to 10 may be implemented by the device for visual field testing 600. The device for visual field testing 600 includes a display module 610, a response module 620 and a processing module 630.

The display module 610 is configured to display, within a preset first duration, a central stimulus point on a detection interface; and is also configured to display, within a preset second duration, a central target and a peripheral target on the detection interface based on a preset target display strategy. The preset target display strategy is used to determine display data of the central target and the peripheral target, and the preset second duration is less than the preset first duration.

The response module 620 is configured to determine a first response result corresponding to the first trigger operation and a target position area of a next round of display of the peripheral target in response to the subject's first trigger operation on the detection interface.

The processing module 630 is configured to determine a testing result of the subject based on first response results corresponding to a preset number of detections, and the testing result includes an attended degree and an attention area.

The various modules of the device for visual field testing 600 may respectively execute the corresponding steps in the above method embodiment, so each module will not be repeated here, and for details, references may be made to the description of the corresponding steps above.

It should be noted that the above device for visual field testing 600 is embodied in the form of a functional module. The term “module” here may be implemented in the form of software and/or hardware, and no specific limitation is imposed on this.

For example, a “module” may be a software program, a hardware circuit, or a combination of the two that implements the above-mentioned functions. The hardware circuit may include an application specific integrated circuit (ASIC), an electronic circuit, a processor (such as a shared processor, a proprietary processor, or a group processor, etc.) and a memory for executing one or more software or firmware programs, a merged logic circuit, and/or other suitable components that support the described functions.

Therefore, the modules of each example described in the embodiments of the present application may be implemented in electronic hardware alone, or in a combination of computer software and electronic hardware. Whether these functions are performed in hardware or software depends on the specific application and design constraints of the technical solution. Professional and technical personnel may use different methods to implement the described functions for each specific application, but such implementation should not be considered to be beyond the scope of the present application.

In the display equipment shown in FIG. 1, the memory may be used to store the relevant program of the method for visual field testing provided in the embodiment of the present application, and the processor may be used to invoke the relevant program of the method for visual field testing stored in the memory when performing image repair on the terminal equipment, and execute the method for visual field testing of the embodiment of the present application.

The present application also provides a computer program product. The method for visual field testing of any method embodiment in the present application is implemented when the computer program product is executed by the processor.

The computer program product may be stored in the memory, for instance, as a program. The program, after going through processing procedures such as preprocessing, compilation, assembly and linking, is ultimately transformed into an executable object file that can be executed by the processor.

The present application also provides a computer-readable storage medium, on which a computer program is stored, and when the computer program is executed by the computer, the method for visual field testing of any method embodiment in the present application is implemented. The computer program may be a high-level language program or an executable object program.

The computer-readable storage medium is, for example, a memory. The memory may be a volatile memory or a non-volatile memory, or the memory may include both a volatile memory and a non-volatile memory. Herein, the non-volatile memory may be a read-only memory (ROM), a programmable ROM (PROM), an erasable PROM (EPROM), an electrically EPROM (EEPROM) or a flash memory. The volatile memory may be a random-access memory (RAM), which is used as an external cache. By way of example but not limitation, many forms of RAM are available, such as static RAM (SRAM), dynamic RAM (DRAM), synchronous DRAM (SDRAM), double data rate SDRAM (DDR SDRAM), enhanced SDRAM (ESDRAM), synch link DRAM (SLDRAM) and direct Rambus RAM (DR RAM).

In the present application, the term “at least one” means one or more, and the term “multiple” means two or more. The “at least one of the following” or similar expressions refers to any combination of these items, including any combination of single items or multiple items. For example, at least one of a, b, or c may be represented by: a, b, c, a-b, a-c, b-c, or a-b-c, wherein a, b, c may be single or multiple.

It should be understood that in various embodiments of the present application, the order of the sequence numbers of the above-mentioned processes does not imply the sequence of execution. The execution order of each process should be determined by its function and internal logic, and should not constitute any limitation on the implementation process of the embodiment of the present application.

It can be realized by a person of ordinary skill in the art that the exemplary units and algorithm steps described in the embodiments disclosed herein may be implemented by electronic hardware, or by a combination of computer software and electronic hardware. Whether these functions are implemented in hardware or software depends on the specific application and design constraints of the technical solution. Professional technicians may adopt different methods to implement the described functions for each specific application, but such implementation should not be considered to be beyond the scope of the present application.

It can be clearly understood by persons skilled in the art that for the convenience and simplicity of description, for the specific working process of the above-described system, device and unit, references may be made to the corresponding process in the aforementioned method embodiment, which thus will not be repeated here.

In the several embodiments provided in the present application, it should be understood that the disclosed device and method may be implemented in other ways. For instance, the device embodiments described above are merely illustrative; for example, the division of units is merely a logical function division, and in actual implementation, there may be other division methods. For example, multiple units or components may be combined or integrated into another system, or some features may be ignored, or not executed. Another point is that the couplings or direct couplings or communication connections that are shown or discussed among the components may be indirect couplings or communication connections through some interfaces, devices or units. These couplings/connections may be in electrical, mechanical or other forms.

The unit described as a separate component may or may not be physically separated, and the component displayed as a unit may or may not be a physical unit, that is, it may be located in one place, or it may be distributed on multiple network units. Some or all of the units may be selected according to actual needs to achieve the purpose of the scheme of this embodiment.

Furthermore, in each of the embodiments of the present application, the various functional units may be integrated into a single processing unit, or these units may be physically present separately, or two or more of these units may be integrated into one unit.

The above description is only some specific implementation of the present application, but the protection scope of the present application is not limited thereto. Any technician familiar with the technical field can easily think of variations or replacements within the technical scope disclosed by this application, and all such variations or replacements should be covered within the protection scope of this application. Therefore, the protection scope of the present application shall be based on the protection scope of the claims.

What is claimed is:

1. A method for visual field testing, applied to a piece of display equipment which comprises a display for presenting a detection interface, and the method comprising:
displaying, within a preset first duration a central stimulus point on the detection interface;
displaying, within a preset second duration, a central target and a peripheral target on the detection interface based on a preset target display strategy, wherein the preset target display strategy is used to determine display data of the central target and the peripheral target;
determining, in response to a first trigger operation of a subject on the detection interface, a first response result corresponding to the first trigger operation and a target position area of a next round of display of the peripheral target; and
determining a testing result of the subject based on first response results corresponding to a preset number of detections, wherein the testing result comprises an attended degree and an attention area.

2. The method for visual field testing according to claim 1, further comprising:
determining, based on the attended degree, a quality indicator area in the detection interface;
displaying the central target and displaying a judgment target in the quality indicator area based on the preset target display strategy within a preset third duration;
determining, in response to a second trigger operation of the subject on the detection interface, a second response result corresponding to the second trigger operation; and
determining, based on a preset number of indicator trials and the second response result, a quality indicator of the subject during testing, wherein the preset number of indicator trials is smaller than the preset number of detections.

3. The method for visual field testing according to claim 2, further comprising:
indicating that the testing result of the subject is inaccurate if a saccade, a missing of the central target, a false negative or a false positive is presented in the quality indicator; and
indicating that the testing result of the subject is accurate if the saccade, the missing of central target, the false negative and the false positive are not presented in the quality indicator.

4. The method for visual field testing according to claim 2, wherein the quality indicator comprises a saccade rate, a missing rate, a false positive rate and a false negative rate, and the method further comprises:
indicating that a saccade is detected during a testing process of the subject if the saccade rate is higher than a preset first threshold;
indicating that a missing of the central target is detected during the testing process of the subject if the missing rate is higher than a preset second threshold;
indicating that a false positive is detected during the testing process of the subject if the false positive rate is higher than a preset third threshold; and
indicating that a false negative is detected during the testing process of the subject if the false negative rate is higher than a preset fourth threshold.

5. The method for visual field testing according to claim 3, wherein the quality indicator comprises a saccade rate, a missing rate, a false positive rate and a false negative rate, and the method further comprises:
indicating that the saccade is detected during a testing process of the subject if the saccade rate is higher than a preset first threshold;
indicating that the missing of the central target is detected during the testing process of the subject if the missing rate is higher than a preset second threshold;
indicating that the false positive is detected during the testing process of the subject if the false positive rate is higher than a preset third threshold; and
indicating that the false negative is detected during the testing process of the subject if the false negative rate is higher than a preset fourth threshold.

6. The method for visual field testing according to claim 1, wherein the preset target display strategy comprises a random algorithm and a target adjustment rule, and wherein the display data comprises a stimulus content, a stimulus angular size and a display position, and said displaying, within the preset second duration, the central target and the peripheral target based on the preset target display strategy further comprises:
determining the stimulus content of the central target and the stimulus content of the peripheral target based on the random algorithm and a preset display set;
determining the stimulus angular size of the central target and the stimulus angular size of the peripheral target based on the target adjustment rule; and
determining the display position of the peripheral target based on the target adjustment rule, the target position area and a preset detection range.

7. The method for visual field testing according to claim 1, further comprising:
dividing the detection interface based on a preset division threshold to obtain target meridians in the detection interface, the target meridians comprise a first meridian; and
displaying, within the preset second duration, the peripheral target on the first meridian.

8. The method for visual field testing according to claim 7, wherein the target meridians further comprise multiple second meridians, the multiple second meridians are different from the first meridian, and the method further comprises:
displaying, within the preset second duration, peripheral stimulus points on the multiple second meridians respectively, wherein the peripheral stimulus points and the peripheral target have a same eccentricity.

9. The method for visual field testing according to claim 1, further comprising:
obtaining initial information of the subject; and
determining a test report of the subject based on the initial information and the testing result, and storing data in the test report of the subject.

10. The method for visual field testing according to claim 9, further comprising:
determining age information of the subject based on the initial information; and
obtaining, based on the age information of the subject, age-matched visual field data corresponding to the age information, and determining comparison data between the testing result and the age-matched visual field data.

11. A device for visual field testing, comprising:
a display module, configured to display, within a preset first duration, a central stimulus point on a detection interface; and further configured to display, within a preset second duration, a central target and a peripheral target on the detection interface based on a preset target display strategy, wherein the preset target display strategy is used to determine display data of the central target and the peripheral target, and the preset second duration is less than the preset first duration;
a response module, configured to determine, in response to a subject's first trigger operation on the detection interface, a first response result corresponding to the first trigger operation and a target position area of a next round of display of the peripheral target; and
a processing module, configured to determine a testing result of the subject based on first response results corresponding to a preset number of detections, and the testing result comprises an attended degree and an attention area.
